(19) United States
(12) Patent Application Publication
(10) Pub. No.: US 2006/0013775 A1
(43) Pub. Date: Jan. 19, 2006

(54) Use of ppar activators for the treatment of pulmonary fibrosis

(76) Inventors: Robert William Gristwood, Cambridge (GB); David Cavalla, Cambridge (GB); Hazel Judith Bardsley, Konstanz (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/495,732

(22) PCT Filed: Nov. 26, 2002
(86) PCT No.: PCT/GB02/05316

§ 371 Date: Jan. 18, 2005

(30) Foreign Application Priority Data
Nov. 26, 2001 (GB) 0128304.3
Jul. 11, 2002 (GB) 0216128.9

(51) Int. Cl. A61L 9/04; A61K 31/573; A61K 31/4439; A61K 31/426

(52) U.S. Cl. 424/45; 514/171; 514/340; 514/369

(57) ABSTRACT

An activator of PPAR gamma is useful for the treatment of pulmonary fibrosis.

### FIELD OF THE INVENTION

This invention relates to a new use for known compounds; and in particular to the therapeutic use of PPAR activators.

### BACKGROUND OF THE INVENTION

Interstitial lung disease (ILD) is a broad category of lung diseases that includes more than 130 disorders which are characterized by scarring of the lungs. ILD accounts for 15% of the cases seen by pulmonologists (lung specialists). Another name for ILD is pulmonary fibrosis. Some of the interstitial lung disorders include: idiopathic pulmonary fibrosis, hypersensitivity pneumonitis, sarcoidosis, eosinophilic granuloma, Wegener's granulomatosis, idiopathic pulmonary hemosiderosis and bronchiolitis obliterans.

Approximately two-thirds of these conditions have no known cause and are therefore termed idiopathic pulmonary fibrosis (IPF). Known causes include: occupational and environmental exposure, inorganic dust (silica, hard metal), organic dust (bacteria, animal proteins), gases, fumes, drugs and poisons, chemotherapy, antibiotics (this is rare), radiation therapy, infections (including residues of active infection of any type), connective tissue disease, systemic lupus erythematosus, rheumatoid arthritis and progressive systemic sclerosis.

The most common symptoms of ILD are shortness of breath with exercise and a non-productive cough. Some people also exhibit fever, weight loss, fatigue, muscle and joint pain, and abnormal chest sounds, depending upon the cause.

ILD is a disease in which tissue in the lungs called the interstitium becomes inflamed or scarred. The interstitium includes a portion of the connective tissue of the blood vessels and alveoli (air sacs) and makes up the membrane where the exchange of oxygen and carbon dioxide takes place. After the inflammation occurs, scarring, or fibrosis, develops. The general pattern is: injury to lung cells, inflammation, and fibrosis. The progression of ILD can vary from person to person, and each person responds differently to treatment. Many doctors characterise ILD in stages, to indicate how much of the affected lung tissue is inflamed and how much is scarred.

The PPARγ receptor is a subtype of the PPAR (peroxisome proliferator-activated receptor) family of nuclear hormone receptors. It has been shown to function as an important regulator in lipid and glucose metabolism, adipocyte differentiation, inflammatory response and energy homeostasis.

The thiazolidinediones rosiglitazone and pioglitazone are used for the treatment of insulin resistance in type 11 diabetes. Thiazolinedione activators of PPARγ have also been shown to have anti-proliferative and anti-inflammatory effects in vascular myocytes and macrophages. Furthermore, troglitazone has been shown to have anti-proliferative effects on keratinocytes in psoriasis. In this disease, keratinocyte hyperproliferation and immune dysfunction are major components. Such compounds and their utility in therapy are described in U.S. Pat. No. 5,594,015, U.S. Pat. No. 5,824,694, U.S. Pat. No. 5,925,657 and U.S. Pat. No. 5,981,586.

Conversely, activators of the alpha subtype of the PPAR (PPARα), which include such compounds as clofibrate and gemfibrozil, have been described in U.S. Pat. No. 6,060,515 for their ability to enhance epithelial barrier development. Acting through an effect on trans-epithelial water loss, hypertrophic scars and keloids are among many skin conditions that are said to be susceptible to such treatment.

Inflammatory leukocytes, for example eosinophils, neutrophils or macrophages, are thought to play a role in the inflammatory component of respiratory diseases.

The use of PPARγ agonists for the treatment of a disease or condition associated with increased numbers of neutrophils and/or neutrophil over-activation is described in WO00/62766.

The use of anti-inflammatory or immunosuppressive agents in the treatment of ILD, asthma or chronic obstructive pulmonary disease (COPD) is well known. These drugs have effects on inflammatory leukocytes, for example reducing their number and/or deactivating them (Baughman et al., Curr. Opinion Pulm. Med. 2001 September; 7(5): 309-313). Such agents include corticosteroids, which are a common option and regarded as the gold standard of anti-inflammatory agents. Despite their effectiveness in controlling inflammation, they do not address other elements of these diseases, including fibrosis.

ILD, asthma and COPD include a range of responses to anti-inflammatory agents such as corticosteroids. Recent data indicate that, following such treatment, less than 30% of IPF patients show objective evidence of improvement (Allen et al, Respir. Res. 2002; 3: 13). Most asthmatic patients respond well to corticosteroids but some are known to be poorly responsive, and it has been suggested that, in such patients, fibrogenesis dominates over inflammation (Bosse et al., Am. J. Respir. Crit. Care Med. 1999 February; 159(2): 596-602). Inhaled corticosteroids are widely prescribed for the treatment of stable COPD, despite lack of proven efficacy, indicating that steroids do not appear to redress the non-inflammatory pathophysiology that is thought to be important in the pathogenesis of this disease (Culpitt et al, Am. J. Respir. Crit. Care Med. 1999 November; 160(5 Pt 1):1635-9).

Recent evidence suggests that lung myofibroblasts play an important role in the progression of pulmonary fibrosis (Uhal et al. 1998, Am. J. Physiol. 275 (Lung Cell. Mol. Physiol. 19): 1192-1199). In particular, these myofibroblasts are capable of inducing death in alveolar epithelial cells and it is believed that accumulating fibroblasts in human lung tissue are found in close proximity to unrepaired or abnormal alveolar epithelium (Uhal et al., supra). Alveolar cells have important antifibrotic functions (Simon et al. 1995, in Pulmonary Fibrosis, ed. Phan & Thrall, New York Dekker vol. 80, pp 511-540) and it may be concluded that myofibroblast actions cause, directly and or indirectly, fibrosis of the lung.

### SUMMARY OF THE INVENTION

Surprisingly, it has been found that an activator of PPAR gamma such as pioglitazone has the ability to reduce numbers of viable lung myofibroblasts and thereby, as explained above, reduce the lung fibrosis. According to the present invention, a PPARγ agonist may be used to treat any form of ILD, including those to which reference is made above. The invention is particularly useful where the condition has a fibrotic component.

The ILD or pulmonary fibrosis that is treated may be a component of another condition, e.g. chronic obstructive pulmonary disease (COPD) or asthma. It may also be the third stage of acute respiratory disease syndrome (ARDS), i.e. following the usual first and second stages of pathology, i.e. damage to epithelial cells, and proliferation.

The invention may involve treatment or prevention of conditions. As an example of the latter, one type of pulmonary fibrosis is associated with drug treatments including bleomycin, amiodarone as well as radiotherapy (in a percentage of patients). This may be treated prophylactically with a PPAR agonist, to prevent the occurrence of fibrosis.

It is now evident that the use of PPARγ agonists for treatment of a fibrotic state, condition or disease of the lung in a host suffering therefrom has not been described before. Neither has the use of PPARγ agonists for treatment of ILD, asthma or COPD in a host wherein the inflammation is adequately treated, e.g. by corticosteroids.

Accordingly, the present invention particularly provides:
the use of a PPARγ agonist for the treatment of ILD, asthma or COPD in a host in need thereof, wherein the host is not in need of anti-inflammatory treatment;
the use of a PPARγ agonist for the treatment of ILD, asthma or COPD in a host in need thereof, wherein the host is not in need of treatment to address the adverse effects of increased numbers of neutrophils and/or neutrophil overactivation in the lung;
the use of a PPARγ agonist for the treatment of ILD, asthma or COPD in a host in need thereof, wherein the host is concurrently treated with an effective dose of a corticosteroid or other anti-inflammatory agent; and
the use of a PPARγ agonist for the treatment of ILD, asthma or COPD in a host in need thereof, wherein the host is concurrently treated with an effective dose of a corticosteroid or other agent to redress the increased numbers of neutrophils and/or neutrophil overactivation in the lung.

### DESCRIPTION OF PREFERRED EMBODIMENTS

Any PPARγ activator may be used in this invention provided it has the desired activity. Well known activators of this receptor include the thiazolidinediones, troglitazone, pioglitazone, rosiglitazone and ciglitazone, isaglitazone, darglitazone and englitazone. It will be understood that a prodrug or metabolite for such a compound can be used. Other non-thiazolidinedione compounds have recently been identified such as the phenyl alkanoic acids described in WO97/31907 and WO00/08002, the oxazoles and thiazoles described in WO99/58510, the oximinoalkanoic acids described in WO01/38325, the benzoic acid derivatives described in WO01/12612, the sulphonamides described in WO99/38845, the β-aryl-α-oxysubstituted alkylcarboxylic acids described in WO00/50414, and the quinolines described in WO00/64876 and WO00/64888. In addition, the natural compound 15-deoxy-γ-12,14-prostaglandin J2 has also been found to be a ligand for PPARγ and to have effects mediated through this receptor (Forman et al, Cell 93(5): 813-819, 1995). Similar effects have also been found for metabolites of 15-deoxy-Δ-12,14-prostaglandin J2 (Kliewer et al, Cell 83(5): 813819, 1995) and for various fatty acids and eicosanoids (Kliewer et al, PNAS USA 94(a): 4318-4323, 1997).

Despite the structural variation tolerated by PPARγ, there is a substantial similarity in biological effect due to activation of this receptor. PPAR agonists share a common binding mode to their receptors. Despite differences in the chemical structure of these agonists, the acidic headgroups of these agonist ligands accept a hydrogen bond from a tyrosine residue in the AF2 helix and/or a histidine or tyrosine residue in helix-5 (see description in WO01/17994). Compounds with the ability to activate PPARγ receptors can be expected to be useful in this invention.

For use in the invention, therapeutic compounds may be administered to human patients topically or by subcutaneous injection. Oral and parenteral administration are used in appropriate circumstances apparent to the practitioner. Preferably, the compositions are administered in unit dosage forms suitable for single administration of precise dosage amounts. Guidance on formulations of this type is provided in WO02/087576 (the content of which, and of all other publications identified herein, is incorporated by reference).

The active agent is preferably administered by inhalation, e.g. to the lower lung. This may be achieved through control of particle properties (including shape, size and electrostatic forces), using a dry powder or liquid particle formulation. Suitable particle sizes are up to 1 μm, or up to 5 μm or above, depending on the intended target.

The dosage of active agent for pulmonary administration can be determined by one skilled in the art, based on factors such as the condition of the patient, the severity of the disease and frequency of administration. It is typically 0.01 mg to 1000 mg.

The concentration of PPARγ activator required to have a maximally effective antifibrotic effect in the lungs may be higher than that which may be safely achieved clinically by administration of the activator via any route other than the inhaled route. For example, maintained free plasma concentrations of pioglitazone following oral administration to man, of conventional clinical dosages, would be expected to be substantially below 10 μM.

The active agent may be provided in a device suitable for pulmonary delivery, for delivery topically to the lung. This can be achieved using a range of pulmonary systems and formulation techniques known to those skilled in the art such as, but not limited to, nebulisers, multi-dose inhalers, dry powder inhalers and pressurised metered multi-dose inhalers. The active agent can be readily formulated for inhalation, e.g. with one or more conventional additives such as carriers, excipients, surface active agents etc.

In addition to the therapeutic compound, the compositions may include, depending on the formulation desired, pharmaceutically acceptable, non-toxic carriers or diluents, which include vehicles commonly used to form pharmaceutical compositions for animal or human administration. The diluent is selected so as not to unduly affect the biological activity of the combination. In addition, the pharmaceutical composition or formulation may include additives such as other carriers, adjuvants or non-toxic, non-therapeutic, non-immunogenic stabilizers and the like.

Furthermore, excipients can be included in the formulation. Examples include cosolvents, surfactants, oils, humectants, emollients, preservatives, stabilizers and antioxidants. Any pharmacologically acceptable buffer may be used, e.g., Tris or phosphate buffers. Effective amounts of diluents, additives and excipients are those which are effective to obtain a pharmaceutically acceptable formulation in terms of solubility, biological activity, etc.

The term “unit dosage form” refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the unit dosage forms of this invention are dictated by and dependent on (a) the unique characteristics of the active material and the particular effect to be achieved, and (b) the limitations inherent in the art of compounding such an active material for use in humans and animals.

Examples of unit dosage forms are tablets, capsules, pills, powder packets, wafers, suppositories, granules, cachets, teaspoonsful, tablespoonsful, droppersful, ampoules, vials, aerosols with metered discharges, segregated multiples of any of the foregoing, and other forms as herein described.

Thus, a composition for use in the invention includes a therapeutic compound which may be formulated with one or more conventional, pharmaceutically acceptable vehicles, preferably for pulmonary administration. Formulations may also include small amounts of adjuvants such as buffers and preservatives to maintain isotonicity, physiological and pH stability. Means of preparation, formulation and administration are known to those of skill. See generally Remington's Pharmaceutical Science 15th ed., Mack Publishing Co., Easton, Pa. (1980).

Slow or extended-release delivery systems, including any of a number of biopolymers (biological-based systems), systems employing liposomes, and polymeric delivery systems, can be utilized with the compositions described herein to provide a continuous or long-term source of therapeutic compound. Such slow release systems are applicable to formulations for topical, ophthalmic, oral, and parenteral use.

Further information of relevance may be found in WO02/087576, including evidence of the utility of PPARγ activators to affect fibroblasts. Evidence on which this invention is more particularly based is in the following Example.

### EXAMPLE

Primary human lung fibroblasts were derived from patients with ILD (Idiopathic Pulmonary Fibrosis or Chronic Hypersensitivity Pneumonitis). Patients had clinical, functional and radiologic features which fulfil the diagnostic criteria for an ILD. Briefly, they had progressive dyspnea, bilateral reticulonodular images on chest roentgenogram, restrictive lung functional impairment, with decreased lung volumes and compliance, and hypoxemia at rest that worsened with exercise.

The methods used to isolate and culture the lung fibroblasts and count cells are described in Wang et al, Am. J. Physiol. Lung 277:L1158-1164 (1999). In brief, lung fibroblasts were isolated by trypsin digestion of tissues minced to 1 mm² fragments. Fibroblast/myofibroblast strains were established in Dulbecco's modified Eagle's medium (or in Hams F-12 medium) supplemented with 10% fetal calf serum, 200 U/ml penicillin, and 200 mg/ml streptomycin, and were cultured in 24-well plates. All cells were cultured at 37° C. in 95% air-5% carbon dioxide. For these experiments, 2 strains were used.

In order to quantify myofibroblast numbers, the myofibroblast marker alpha-smooth muscle actin (α-SMA) was measured. Detection of α-SMA was achieved with a fluorescent (FITC) monoclonal antibody specific for α-SMA applied to ethanol-fixed cells (see Wang et al., referenced above).

In a first experiment, the 2 strains were grown to 70-80% confluence. The cells were exposed to pioglitazone at 3 μM or drug vehicle for 3 days, after which the number of α-SMA positive cells was quantified (sample size 24) as a percentage of total cells. For one strain, the percentage of α-SMA cells was 27 (standard error mean 3.7) with control and 17 (standard error mean 2.5) in the presence of 3 μM pioglitazone. The drug effect was statistically significant (P≦0.01 Student-Newman-Keuls Multiple Comparisons Test). For the second strain, the respective values were 30.6 (standard error of mean 2.7) and 26 (standard error of mean 2.9) although the difference was not statistically significant.

In a second experiment, the effect of pioglitazone on the second strain was studied again, but the exposure time was increased to 10 days and the effects of lower and higher concentrations (1 μM and 10 μM) were studied. After 10 days treatment with vehicle, the percentage of α-SMA cells was 16.5 (standard error of mean 2.7); after 10 days treatment with pioglitazone at 1 μM the percentage was 15.3 (standard error of mean 1.8); and after treatment with pioglitazone at 10 μM the percentage of α-SMA cells was 7.4 (standard error of mean 1.8) (all n=4). The reduction in α-SMA cells by 10 μM pioglitazone was significant (P≦0.05 Dunnett Multiple Comparisons Test). In this experiment, there were no significant changes in total cell numbers.

These data clearly establish the ability of pioglitazone to reduce numbers of human lung myofibroblasts.

1. A method for treating pulmonary fibrosis wherein said method comprises administering, to a patient in need of such treatment, an activator of PPARy.

2. The method, according to claim 1, wherein the activator is a thiazolinedione.

3. The method, according to claim 1, wherein the activator is pioglitazone.

4. The method, according to claim 1, wherein the activator of PPARy is administered by inhalation.

5. The method, according to claim 1, wherein the pulmonary fibrosis is associated with COPD.

6. The method, according to claim 1, wherein the pulmonary fibrosis is associated with asthma.

7. The method, according to claim 1, wherein the pulmonary fibrosis is associated with acute respiratory distress syndrome (ARDS).

8. The method, according to claim 7, wherein the pulmonary fibrosis is associated with the third stage of ARDS.

9. The method, according to claim 1, used for the treatment of pulmonary fibrosis in a patient who is undergoing chemotherapy.

10. The method, according to claim 1, used for the treatment of pulmonary fibrosis in a patient who is undergoing radiation therapy.

11. The method, according to claim 1, used for the treatment of pulmonary fibrosis in a patient who is undergoing therapy with amiodarone.

12. The method, according to claim 1, used for the treatment of idiopathic pulmonary fibrosis.

13. The method, according to claim 1, used for the treatment of pulmonary fibrosis in a patient who is undergoing therapy with an anti-inflammatory agent.

14. The method, according to claim 13, wherein the anti-inflammatory agent is corticosteroid.

15. The method according claim 1, used for the treatment of pulmonary fibrosis in a patient who is resistant to treatment with corticosteroids.

16. A formulation for inhalation, comprising an activator of PPARy.

17. A device for pulmonary delivery, comprising activator of PPARy.

18. The formulation, according to claim 16, wherein the activator is a thiazolinedione.

19. The formulation, according to claim 16, wherein the activator is pioglitazone.

20. The device, according to claim 17, wherein the activator is a thiazolinedione.

21. The device, according to claim 17, wherein the activator is pioglitazone.
